(19) United States
(12) Patent Application Publication
(10) Pub. No.: US 2012/0121616 A1
(43) Pub. Date: May 17, 2012

(54) IMMUNE BALANCE-REGULATING AGENT

(76) Inventors: Takashi Nishimura, Sapporo (JP); Tsutomu Kano, Tokyo (JP)

(21) Appl. No.: 13/126,612

(22) PCT Filed: Aug. 3, 2010
(86) PCT No.: PCT/JP2010/063049

§ 371 Date: Apr. 28, 2011

(30) Foreign Application Priority Data
Aug. 6, 2009 (JP) 2009-183696

(51) Int. Cl. A61K 39/00; A61P 35/00; A61P 37/02; A61P 31/00

(52) U.S. Cl. 424/184.1

(57) ABSTRACT

Disclosed is a novel use of superheated stream-treated material. Disclosed is an immune balance-regulating agent comprising a superheated steam-treated product of crown daisy (Chrysanthemum coronarium). This immune balance-regulating agent, which comprises, as the active ingredient, a superheated stream-treated product of crown daisy that has been used as a food for a long time, is a composition having a very high safety and exerting an effect of controlling and normalizing the immune balance of a living organism. The present invention has been completed based on the finding that the superheated steam-treated product of crown daisy has anti-infective and antitumor activities based on type-1 immune stimulating effect and, moreover, shows an effect of ameliorating allergic diseases, which are caused by excessive type 2 immune responses, by controlling immune balance.

### CROSS REFERENCE TO RELATED APPLICATIONS

This is a U.S. national application of International Application PCT/JP2010/063049, filed Aug. 3, 2010, which claims priority to Japanese Application No. 2009-183696, filed Aug. 6, 2009, the contents of each of which are incorporated by reference in their entireties for all purposes.

### FIELD OF THE INVENTION

The present invention relates to an immune balance regulating agent comprising a preparation obtained by superheated steam treatment of garland chrysanthemum (crown daisy).

### BACKGROUND OF THE INVENTION

Superheated vapor is a vapor which is heated at or above a temperature at which vapor and liquid can co-exist keeping equilibrium under a constant pressure, and for example, steam which is heated at or above 100° C. at 1 atm is called superheated steam. Technology utilizing superheated steam has extended to the fields of sterilization, drying, food processing and the like; technical developments have been carried out, which utilize the advantage of superheated steam treatment of not changing the quality such as color, flavor, taste, texture of food materials in the field of food processing among others

Superheated steam treatment does not change the quality of food materials (Patent Literatures 3 and 4) and has effects of reducing undesirable excessive oils and fats and odor components as well. Furthermore, its utilization has also been advanced as a technology to enhance desired components, and for example, a quercetin-containing composition obtained by superheated steam treatment of quercetin glucoside-containing materials such as onion skin is disclosed in Patent Literature 5, and it is disclosed in Patent Literature 6 that superheated steam treatment of coffee beans provides roasted coffee beans with a decreased content of acrylamide and an increased contents of chlorogenic acids.

Although it is thus expected to obtain a new material in which some physiological functions are provided or enhanced, a material with satisfactory physiological functions has not yet been obtained.

### SUMMARY OF THE INVENTION

An object of the present invention is to provide a new application of a preparation obtained by superheated steam treatment.

The present inventors have found that a preparation obtained by superheated steam treatment of garland chrysanthemum (crown daisy) exhibits an effect of alleviating allergic diseases caused by excessive type 2 immune response by regulating the immune balance, in addition to infection preventive and anti-tumor activities due to the effect of stimulating type 1 immunity, and completed each of the following inventions.

(1) An immune balance regulating agent containing a preparation obtained by superheated steam treatment of garland chrysanthemum.

(2) The immune balance regulating agent according to (1), which is used for anti-infectious disease.

(3) The immune balance regulating agent according to (1), which is used for anti-tumor.

(4) The immune balance regulating agent according to (1), which is used for enhancing type 1 immune system function.

(5) The immune balance regulating agent according to (4), which is used for dendritic cell activation.

(6) The immune balance regulating agent according to (4), which is used for promoting IFN-γ and/or interleukin (IL)-12 production.

The immune balance regulating agent of the present invention is an extremely highly safe composition having an effect of regulating and normalizing the immune balance of a living body, comprising a preparation obtained by superheated steam treatment of garland chrysanthemum, which has been utilized as food from a long time ago, as an active component.

### BRIEF DESCRIPTION OF THE DRAWINGS

FIG. 1 is a diagram showing the upper and lower graphs show an effect of IFN-γ inducing production (promoting production) of the preparations obtained by superheated steam treatment of Example and Comparative Example 1, respectively. The longitudinal axis indicates IFN-γ production level and the abscissa axis indicates the preparations obtained by superheated steam treatment added. FIG. 2 is a diagram showing the influence of IL-12 on the induction of IFN-γ production from spleen cells by the preparation obtained by superheated steam treatment of Example. The longitudinal axis indicates the IFN-γ production level, and the abscissa axis indicates groups without and with the preparation obtained by superheated steam treatment of Example added (a group without addition and a group with garland chrysanthemum added), respectively.

FIG. 3 is a graph depicting flow cytometric results demonstrating the dendritic cell activation effect of the preparation obtained by superheated steam treatment of Example. The abscissa axis indicates the expression level of the measurement target molecule on the cell surface; a group without addition denotes a group without the preparation obtained by superheated steam treatment of Example added and a group with garland chrysanthemum added denotes a group with the preparation obtained by superheated steam treatment of Example added, respectively.

FIG. 4 is a diagram showing an IL-12 production induction ability, which demonstrates the dendritic cell activation effect of the preparation obtained by superheated steam treatment of Example. The longitudinal axis indicates the IL-12 production level and the abscissa axis indicates groups without and with the preparation obtained by superheated steam treatment of Example added (a group without addition and a group with garland chrysanthemum added), respectively.

FIG. 5 is a diagram showing that the preparation obtained by superheated steam treatment of Example demonstrates, TLR (Toll Like Receptor)—dependently, an IFN-γ production inducing (production promoting) effect. The longitudinal axis indicates the IFN-γ production level and the abscissa axis indicates spleen immune cells of a 7-week-old C57BL/6 female mouse (wild type), spleen immune cells of a TLR2-deficient mouse, spleen immune cells of a TLR4-deficient mouse, and spleen immune cells of a TLR9-deficient mouse in the groups without and with the preparation obtained by superheated steam treatment of Example added (a group without addition and a group with garland chrysanthemum added), respectively.

FIG. 6 is a graph depicting the results of the measurement of IFN-γ production in NK1.1-positive and TCR β-negative cells, NK1.1-positive and TCR β-positive cells, CD4-positive cells and CD8-positive cells, without and with the preparation obtained by superheated steam treatment of Example added (a group without addition and a group with garland chrysanthemum added), respectively, by flow cytometry using an intracellular staining method. The longitudinal axis of each graph indicates the expression level of the target molecule of measurement on each cell surface, respectively and the abscissa axis indicates the IFN-γ production level.

FIG. 7 is diagram showing IFN-γ production levels in spleen immune cells of a 7-week-old C57BL/6 female mouse (control) and spleen immune cells of a 7-week-old C57BL/6 female mouse having no NK1.1-positive cells when preparation obtained by superheated steam treatment of Example is not added (a group without addition) and added (a group with garland chrysanthemum added). The longitudinal axis indicates the IFN-γ production level and the abscissa axis indicates respective cells in the groups without and with the preparation obtained by superheated steam treatment of Example added (a group without addition and a group with garland chrysanthemum added).

FIG. 8 is a diagram showing IFN-γ production inducing (production promoting) effect of the preparation obtained by superheated steam treatment of Example (garland chrysanthemum nepurée) and the preparation obtained by ordinary heat treatment of Comparative Example 2 (garland chrysanthemum purée). The longitudinal axis indicates the IFN-γ production level and the abscissa axis indicates groups without and with the preparation obtained by superheated steam treatment of Example added (a group without addition and a group with garland chrysanthemum added), respectively.

### DETAILED DESCRIPTION OF THE INVENTION

The present invention is an immune balance regulating agent containing a preparation obtained by superheated steam treatment belonging to Chrysanthemum Asteraceae, leaves and stems of which are generally considered to be edible and widely distributed domestically in Japan as a commonly ingested vegetable.

In carrying out the present invention, any edible garland chrysanthemum can be used, and for example, the species termed as Chrysanthemum coronarium in nomenclature can be used.

It should be noted that garland chrysanthemum is known to contain a plenty of vitamin C and carotene as nutrients but nothing is known with regard to its immunoregulating effect.

The superheated steam treatment is carried out using garland chrysanthemum as it is or after ground in to an adequate size. The garland chrysanthemum may be raw or dried.

The temperature of steam used for the superheated steam treatment ranges preferably from approximately 120° C. to 500° C., more preferably from 230° C. to 28020 C. The time for the superheated steam treatment will be set appropriately depending upon the size and quantity of a material, and the time ranging approximately from 30 seconds to 240 seconds is preferable in order for the function of immune balance regulating agent of the present invention to be satisfactory.

In addition, the superheated steam treatment may be carried out twice or more with the condition kept the same or changed with regard to the temperature or time condition; furthermore, a grinding process may be incorporated between two or more superheated steam treatments as described in the above-described Patent Literature 1.

A material after superheated steam treatment can be utilized not only as it is for the immune balance regulating agent of the present invention, but also can be used after further treatment such as solid/liquid separation by centrifugation or filtration, extraction using a solvent such as water, alcohols such as ethanol and a mixture thereof, and drying such as spray drying and freeze drying; all of them are called herein “preparation obtained by superheated steam treatment”.

Immune balance regulation, that is, the immune balance regulating effect in the present invention, means an effect which resolves the state in which either one of a type 1 immune system function or a type 2 immune system function, especially the type 2 immune system function is enhanced, and leads to the state where the both immune system functions are regulated. Regulation of immune balance meant in the present invention is used interchangeably with modulation or adjustment of immune balance.

Generally, the type 1 immune system is understood as an immune system involving Th1 cells (type 1 helper T cells) induced by the presentation of an antigenic peptide from dendritic cells and/or macrophages which are antigen-presenting cells and by the effects of IL-12 and/or IFN-γ. Th1 cells produce IL-2, TNF-α, etc., in addition to cytokines such as IFN-γ which suppress the production of IgE antibody through the inhibition of differentiation of Th2 cells (type 2 helper T cells) and the inhibition of maturation of B cells to activate cell-mediated immunity such as killer T cells and enhance the activity of antigen-presenting cells such as dendritic cells and macrophages. On the other hand, the type 2 immune system is understood to be an immune system involving Th2 cells induced by the presentation of an antigenic peptide from macrophages that are antigen-presenting cells and by the effect of IL-4. Th2 cells produce IL-5, IL-6 and IL-10 in addition to cytokines such as IL-4 and IL-13 which enhance the production of antibodies such as IgE through the maturation of B cells and activate humoral immunity.

It is known that IL-4 and IL-10 produced from Th2 cells control the effect of each other to suppress the production of IFN-γ from Th1 cells. It is believed that if the type 2 immune system function is predominant, cell-mediated immunity is suppressed and an infectious disease tends to be serious, and further, IgE antibody production through the maturation of B cells increases, likely leading to allergic predisposition. Therefore, breaking of the balance of the type 1 immune system function and the type 2 immune system function, particularly, excessive enhancement or dominance of the type 2 immune system function is not always preferable for a living body.

The preparation obtained by superheated steam treatment used in the present invention exhibits effects of activating dendritic cells and natural killer cells (NK cells), natural killer T cells (NKT cells), and inducing or promoting the production of IFN-γ and IL-12. The effects of activating NK cells and NKT cells may include, for example, an effect of inducing the production of IFN-γ in NK cells and NKT cells. Thus, by administering the immune balance regulating agent of the present invention to an individual having an enhanced type 2 immune system function among others, the type 1 immune system function can be enhanced, resulting in regulation of the immune balance. In this way, the preparation obtained by superheated steam treatment used in the present invention can be utilized as a type 1 immune system function enhancer to enhance the type 1 immune system function, as well as a dendritic cell activator, an NK cell activator, an NKT cell activator, an IFN-γ production promoter, and an IL-12 production promoter.

In addition, a physiological activity presented by the above mentioned preparation obtained by superheated steam treatment used in the present invention has been confirmed, quite unexpectedly, to be very strong compared to a case where ordinary heat treatment is carried out using the same material.

Furthermore, the preparation obtained by superheated steam treatment used in the present invention can alleviate a condition in which the type 2 immune system function is dominant, for example, allergy, by enhancing the type 1 immune system function, or it is effective in the treatment of diseases such as infectious diseases and malignant tumors in which enhancement of cell-mediated immunity is required, in addition to the treatment of allergic diseases, because it can induce the production of IFN-γ in NK cells and NKT cells to yield an infectious disease suppressing effect and an anti-tumor effect. In other words, a preparation obtained by superheated steam treatment used in the present invention can be utilized as an allergy, an inhibitor, an infectious disease inhibitor, and an anti-tumor agent.

In addition, it can be expected that the preparation obtained by superheated steam treatment used in the present invention has effects of balancing the type 1 immune system function and the type 2 immune system function usually by its oral application, enhancing resistance against the invasion of foreign matters such as infectious diseases, and further, alleviating allergy and autoimmune diseases that are excessive immune response.

Specifically, the prevention, treatment or effect of improving symptoms of infectious diseases with viruses or bacteria, tumors, inflammation, allergic diseases such as atopic dermatitis, skin roughness, sensitive skin, pollinosis, asthma, bronchial asthma, rhinitis, urticaria, and the like can be expected for the immune balance regulating agent of the present invention.

The preparation obtained by superheated steam treatment of the present invention can be used as an immune balance regulating agent as it is, as well as for a pharmaceutical composition such as a prophylactic, suppressive or therapeutic agent for infectious diseases, an anti-tumor agent, and a prophylactic, suppressive or therapeutic agent for allergic diseases. In addition, it can be combined with common excipients to prepare a composition and the composition can be further formulated into common dosage forms of external use for skin, oral formulations, injections, and others.

The above-mentioned compositions or various dosage forms may be provided in a form of drug or quasi drug, by incorporating pharmaceuticals such as vitamins, galenicals, anti-inflammation agents, antihistamic agents, etc., as an active component, if necessary, in addition to the preparation obtained by superheated steam treatment.

As the excipients used in formulation, for example, ingredients widely known and used by those skilled in the art for each dosage form of a solid oral formulation such as a tablet or a capsule, a liquid internal formulation such as aqueous liquid or suspension, ointment, patch, lotion, cream, spray, suppository, etc., can be used in a proper combination with each other.

The amount of the preparation obtained by superheated steam treatment incorporated in the above mentioned composition or dosage form is not specified, somewhat different depending upon the type of dosage form, quality and the degree of expected effect, may be from 1 to 99% by weight, preferably from 10 to 99% by weight, more preferably from 50 to 99% by weight as a dry solid in the total amount of the composition or formulation.

The immune balance regulating agent of the present invention may be formed into a beverage such as a juice or a milk beverage, a dairy product such as yogurt or ice cream, and foods such as soup, jelly, jam, confectionery or breads as it is or in combination with a proper component for beverages or foods; further, it may be formed into a health food or supplement. When the immune balance regulating agent is ingested or administered in the combination with a food, it can be mixed with an excipient, a filler, a binder, a thickener, a emulsifier, a coloring agent, a flavor, a food additive, a condiment or the like, as appropriate, and formed into powder, granules, and tablets depending upon the intended use. Furthermore, it can be ingested by being mixed in a raw material of food to prepare a food, and commercialized as a functional food.

Since the raw material of the preparation obtained by superheated steam treatment used in the present invention is food, the amount of ingestion in a form as the above-mentioned beverage or food is not particularly restricted. An ingestion amount within the range of being ordinarily used as food is desirable, and specifically the amount is from 0.5 to 250 g, preferably from 1 to 200 g per ingestion, and the total amount of ingestion per day is from 0.5 to 500 g, preferably from 1 to 400 g.

In the following, the present invention will be described in more details with reference to Example, but it should not be construed that the present invention be restricted by such an Example.

### EXAMPLE

### Example

Three kg of garland chrysanthemum (Chrysanthemum coronarium) cut to a length of 4 cm was superheat-treated with a high temperature steam under atmospheric pressure for 10 minutes. Garland chrysanthemum after the treatment was treated with “high speed planetary mixer NewTon UM-N13” made by NAGATA SEIKI CO., LTD. at 1100 rpm, for 100 seconds. The garland chrysanthemum treated with the mixer was treated with an ultracentrifuge (SCR2OBA: Hitachi, Ltd.) at 2000 revolution (25,000×g) for 10 minutes to obtain a precipitated fraction and a supernatant fraction, which supernatant fraction was dried using a freeze dryer to prepare a water soluble fraction. Then, the precipitated fraction was suspended in 10 times volume of a 30% by volume ethanol aqueous solution and after stirred for 30 minutes, separated using a filter paper (Whatman Ltd.) into a solid component from the 30% by volume ethanol aqueous solution and a filtrate from the 30% by volume ethanol aqueous solution. After the filtrate from the 30% by volume ethanol aqueous solution was treated with a concentration centrifuge (EYELA) and evaporated, an extracted fraction from the 30% by volume ethanol aqueous solution was prepared by cooling with liquid nitrogen and complete removal of the solvent with a freeze dryer. Subsequently, after the solid component from the 30% by volume ethanol aqueous solution was suspended in 10 times volume of a 60% by volume ethanol aqueous solution and stirred for 30 minutes, it was separated into solid component from the 60% by volume ethanol aqueous solution and filtrate from the 60% by volume ethanol aqueous solution using filter paper (Whatman Ltd.). An extracted fraction from the 60% by volume ethanol aqueous solution was prepared by treating the filtrate from 60% by volume ethanol aqueous solution in a similar manner to the filtrate from the 30% by volume ethanol aqueous solution.

### Comparative Example 1

An extracted fraction from the each 30% by volume ethanol aqueous solution was obtained in the same way as Example except that garland chrysanthemum of Example was replaced with carrot (Daucus carota), tomato (Solanum lycopersicum), spinach (Spinacia oleracea), or onion (Allium cepa).

### Comparative Example 2

After 2 L of water was placed in a relatively large pot and completely boiled, 100 g of garland chrysanthemum was added and heated for 3 minutes. Garland chrysanthemum after heated was thoroughly ground with ACE HOMOGENIZER (AM-3/KN3325012; NIHONSEIKI KAISHA LTD.). After that, ultracentrifugation and ethanol extraction were carried out in a similar manner to Example to obtain an extracted fraction from the 30% by volume ethanol aqueous solution.

### Test Example

### (1) IFN-γ Production Inducing (Production Promoting) Effect

A spleen was taken from a 7-week-old C57BL/6 female mouse purchased from Charles River Inc. The spleen was loosened using tweezers in an RPMI-1640 medium (Wako Pure Chemical Industries, Ltd.) comprising 10% FCS, 2.38 mg/mL Hepes, 0.11 mg/mL sodium pyruvate, 200 U/mL penicillin G, and 0.1 mg/mL streptomycin. Cells were passed through nylon mesh (Wako Pure Chemical Industries, Ltd.) together with the culture and recovered with the tissue part being removed. After the centrifugation treatment using a small cooling centrifuge (himac CF7D2, Hitachi, Ltd.) at 1500 rpm for 5 minutes, the supernatant was discarded, and the sediment was incubated with 2 mL of 0.155 M ammonium chloride at 37° C. for 1 minute and 30 seconds to eliminate erythrocytes and to prepare spleen immune cells/the RPMI-1640 medium. Each of extracted sample obtained in Example and Comparative Example 1 was co-cultured from the concentration of 200 μg/mL, the culture being carried out using a carbon dioxide gas incubator at 37° C. under 5% CO₂ atmosphere. After 48 hours, the supernatant of the culture was recovered and the IFN-γ amount in the culture supernatant was determined using ELISA Mouse IFN-γ BD Opt EIA set (BD Biosciences).

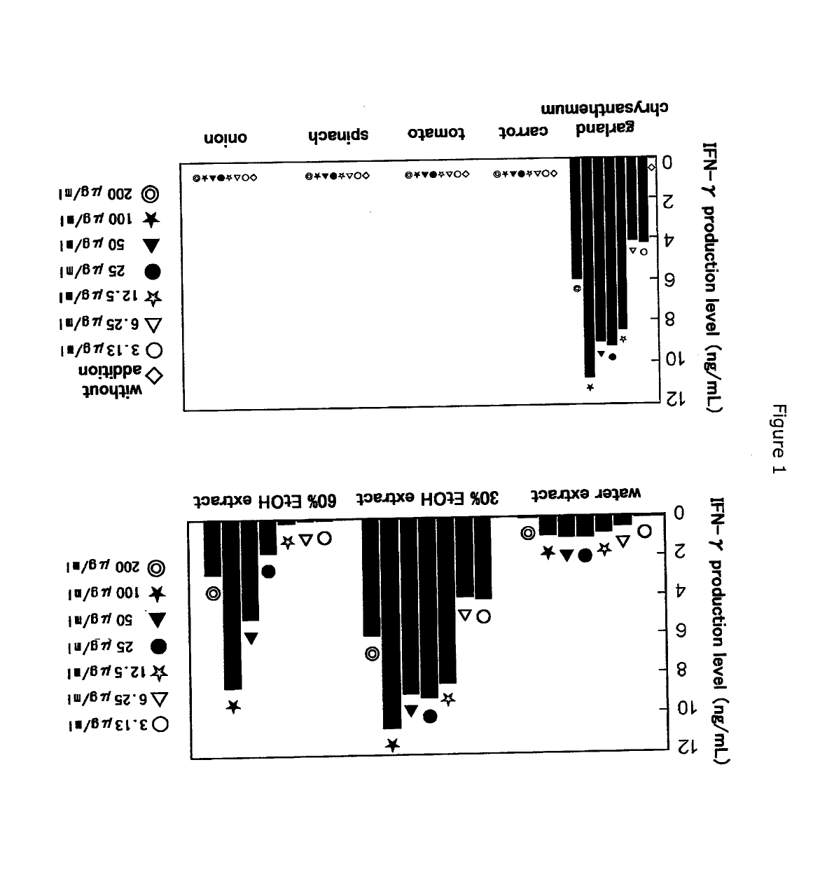

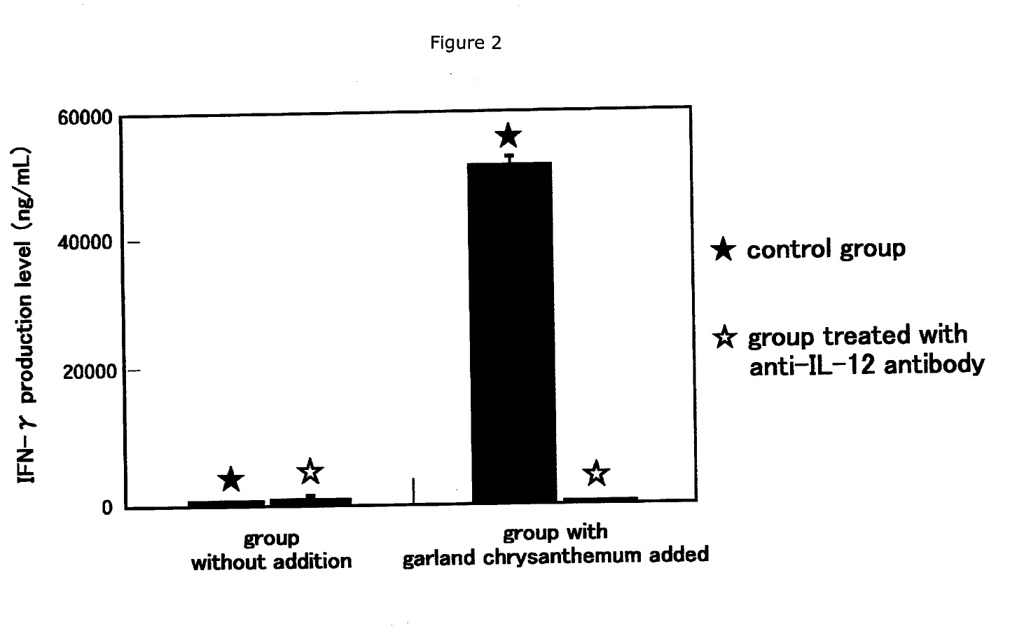

The results are shown in FIG. 1. Only the extract from garland chrysanthemum with 30% by volume ethanol of Example demonstrated a strong activity of inducing IFN-γ production.

(2) Effect of IL-12 on the Induction of IFN-γ Production from Spleen Cells.

A similar experiment to (1) was carried out using the extract from garland chrysanthemum with 30% by volume ethanol of Example, except that the function of IL-12 was inhibited by adding a monoclonal anti-IL-12 antibody in the culture of spleen cells.

The results are shown in FIG. 2. It was confirmed that IFN-γ production induction by garland chrysanthemum was strongly suppressed by the addition of anti-IL-12 antibody, and thus it was shown that IFN-γ production was induced by IL-12.

### (3) Dendritic Cell Activation Effect

Bone marrow cells were collected from the femora of a 7-week-old C57BL/6 female mouse purchased from Charles River Inc., seeded in a 6-well flat bottom plate (Nunc) to be 1×10⁶ cells/well, and cultured in the presence of 10 ng/mL of GM-CSF (PeproTech Inc.) for 6 days to induce dendritic cells that are antigen-presenting cells. These cells and the extract from garland chrysanthemum with the 30% ethanol of Example were co-cultured an in RPMI-1640 medium containing 10% FCS, 2.38 mg/mL Hepes, 0.11 mg/mL sodium pyruvate, 200 U/mL penicillin G, and 0.1 mg/mL streptomycin. Expression levels after 24 hours of MHC class I molecules, MHC class II molecules, CD40 molecules and CD86 molecules on the cell surface were detected by flow cytometry (FACS Calibur; BD Biosciences) using an anti-MHC class I molecule antibody (AF6-88.5), an anti-MHC class II molecule antibody (AF6-88.5), an anti-CD40 antibody (3/23) and an anti-CD86 antibody (GL1).

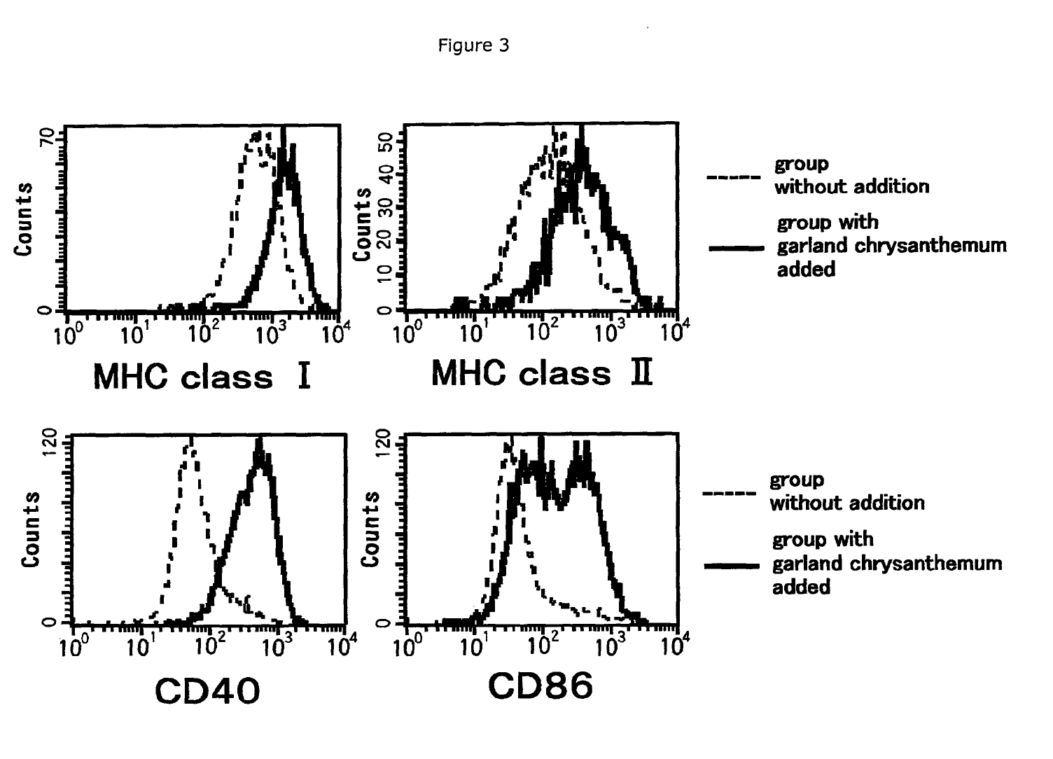

The results are shown in FIG. 3. In the groups in which the extract from garland chrysanthemum with 30% by volume ethanol was added, a significant increase in expression of MHC class I molecules, MHC class II molecules, CD40 molecules and CD86 molecules was observed, compared to the control without addition. From this, the extract from garland chrysanthemum with 30% by volume ethanol was found to activate dendritic cells.

(4) Ability of Inducing IL-12 Production from Dendritic Cells

IL-12 production using the extract from garland chrysanthemum with 30% by volume ethanol of Example was studied in the same condition as (3). The amount of IL-12 p70 contained in the culture supernatant in recovering cells was determined using ELISA Mouse IL-12 p70 BD Opt EIA set (BD Biosciences).

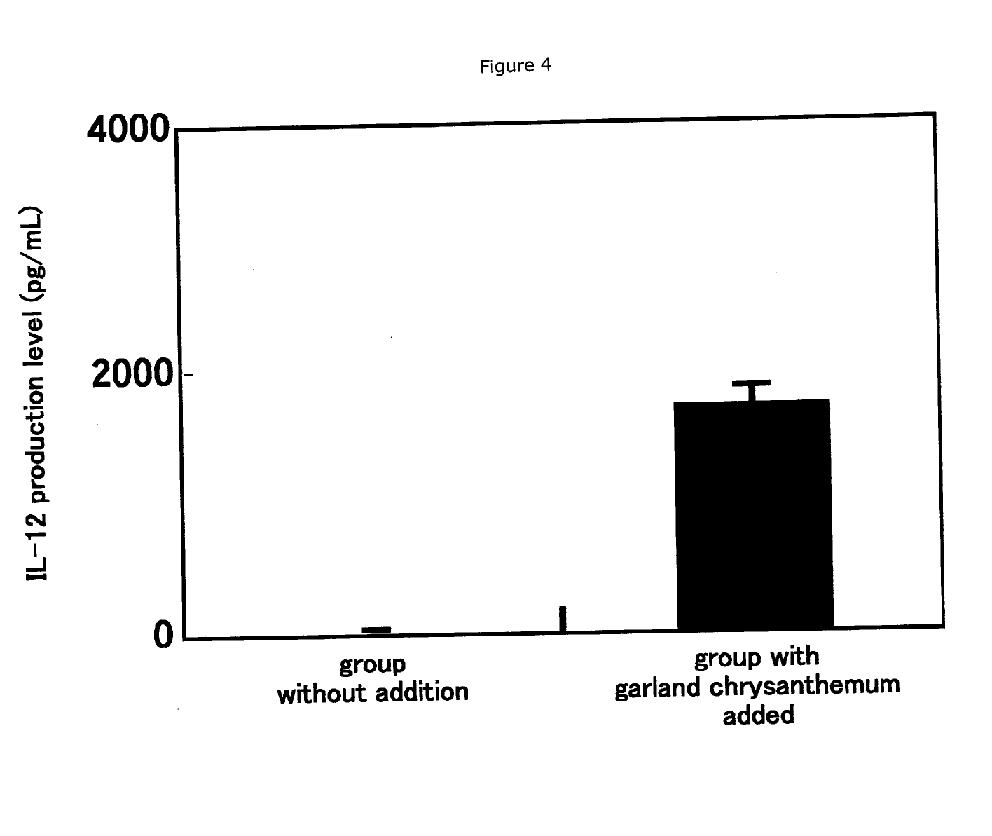

The results are shown in FIG. 4. It was confirmed that the extract from garland chrysanthemum with 30% by volume ethanol induced IL-12 production from dendritic cells.

### (5) Study of TLR Dependency in the Induction of IFN-γ Production

A spleen was collected from a 7-week-old C57BL/6 female mouse purchased from Charles River Inc., or a TLR2 (Toll Like Receptor 2)-deficient mouse, a TLR4 (Toll Like Receptor 4)-deficient mouse and a TLR9 (Toll Like Receptor 9)-deficient mouse obtained from Oriental BioService, Inc., and an experiment was carried out using the extract from garland chrysanthemum with 30% by volume ethanol of Example in the same condition as (1).

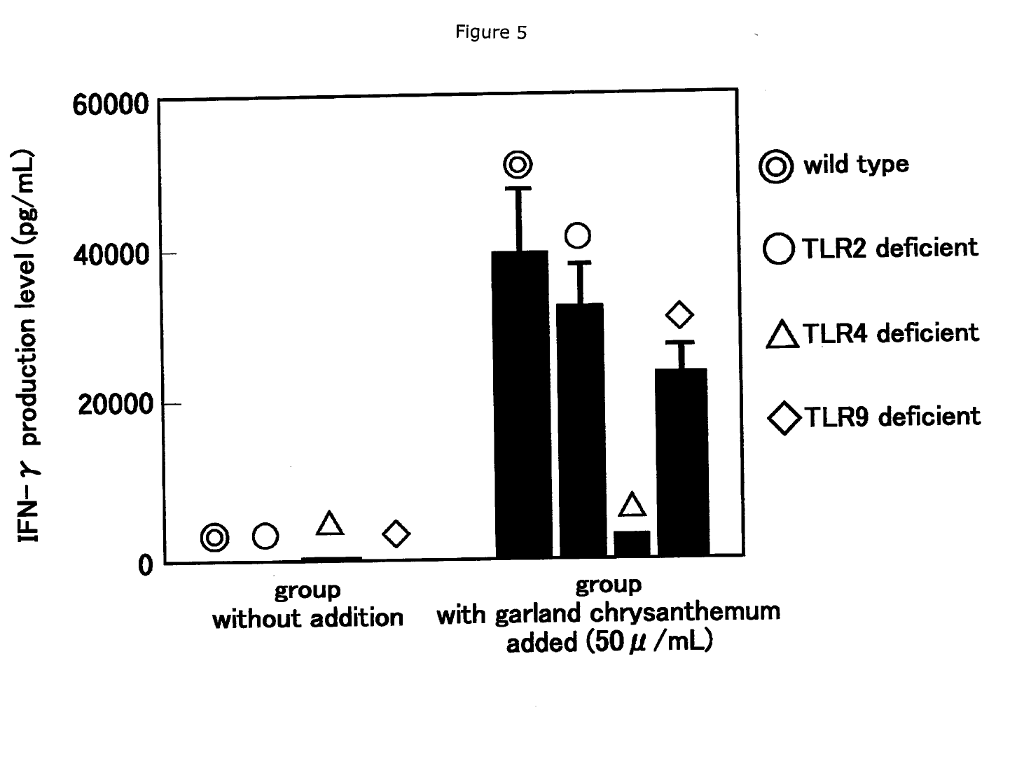

The results are shown in FIG. 5. From the fact that induction of IFN-γ production from spleen cells by garland chrysanthemum was hardly observed when TLR4 was deficient, and attenuated when TLR9 was deficient, it has been revealed that the immune balance regulating effect by garland chrysanthemum is dependent strongly upon TLR4 and partly upon TLR9.

### (6) Identification of IFN-γ Production Inducing Cell

Spleen immune cells/RPMI-1640 medium were prepared in the same way as (1). To this, 25 μg/mL of the extract from garland chrysanthemum with 30% by volume ethanol of Example was added, and cultured using a carbon dioxide gas incubator at 37° C. under 5% CO₂ atmosphere for 12 hours. After Brefeldin A (BFA) was added and an additional 12 hours elapsed, cells were recovered and reacted with an anti-TCR β antibody, an anti-CD4 antibody (GK1.5), an anti-CD8 antibody (53-6.7), an anti-NK1.1 antibody (PK136) and an anti-IFN-γ antibody (XMG1.2), and examined to detect IFN-γ production in NK1.1-positive and TCR β-negative cells, NK1.1-positive and TCR β-positive cells, CD4-positive cells and CD8-positive cells, by flow cytometry (FACS Calibur; BD Biosciences) using an intracellular staining method.

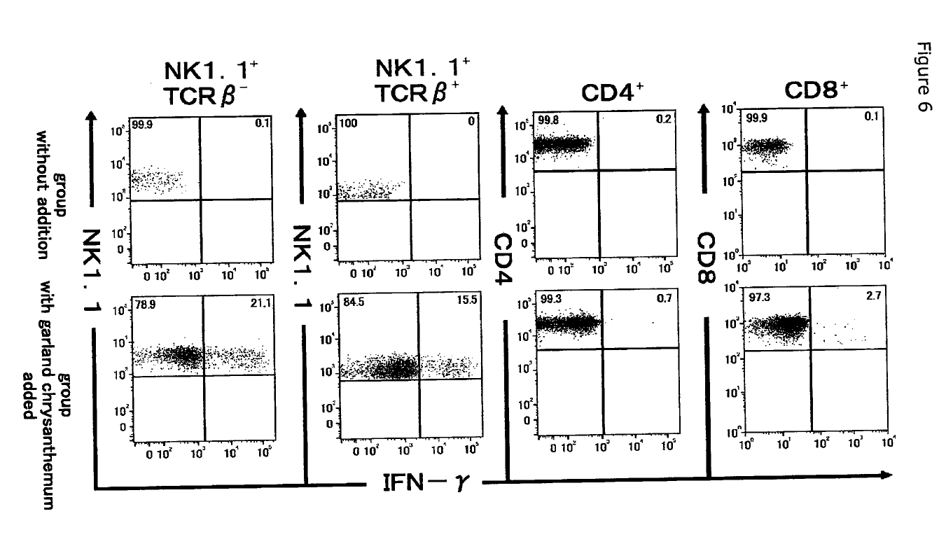

The results are shown in FIG. 6. NK1.1-positive and TCR β-negative cells are found to be NK cells due to expressing the marker of NK cells but not T cell specific marker, while NK1.1-positive and TCR β-positive cells are found to be NKT cells due to expressing the marker of NK cells as well as T cells specific marker. Further, it is revealed that NK1.1-positive and TCR β-negative cells and NK1.1-positive and TCR β-positive cells are activated by the addition of the extract from garland chrysanthemum with 30% by volume ethanol to induce IFN-γ production. From these facts, it has been shown that cells from which IFN-γ production is induced by the addition of the extract from garland chrysanthemum with 30% by volume ethanol are NK cells and NKT cells.

### (7) Confirmation of IFN-γ Production Induction in NK Cells and NKT Cells

From the result of (6), an experiment for confirming IFN-γ production induction in NK cells and NKT cells by the addition of the extract from garland chrysanthemum with 30% by volume ethanol was further carried out.

[7-1]

200 μg of an anti-NK1.1 antibody (PK136) was administered into the peritoneal cavity of a 7-week-old C57BL/6 female mouse purchased from Charles River Inc., and after 24 hours elapsed, the spleen was taken. After that, spleen immune cells/RPMI-1640 medium were prepared in the same way as (1), and after confirmed that NK1.1-positive cells, namely NK cells and NKT cells were not contained, 25 μg/mL of the extract from garland chrysanthemum with 30% by volume ethanol of Example was added and cultured using a carbon dioxide gas incubator at 37° C. under 5% CO₂ atmosphere for 48 hours. Subsequently, the culture supernatant was recovered, and the amount of IFN-γ in the culture supernatant was determined using ELISA Mouse IFN-γ BD Opt EIA set (BD Biosciences).

[7-2]

Spleen immune cells/RPMI-1640 medium were prepared in the same way as [7-1] except that an anti-NK1.1 antibody (PK136) was not administered, and to this added was 25 μg/mL of the extract from garland chrysanthemum with 30% by volume ethanol of Example, and cultured using a carbon dioxide gas incubator at 37° C. under 5% CO₂ atmosphere for 48 hours. Subsequently, the culture supernatant was recovered, and the amount of IFN-γ in the culture supernatant was determined using ELISA Mouse IFN-γ BD Opt EIA set (BD Biosciences), this being taken as a control.

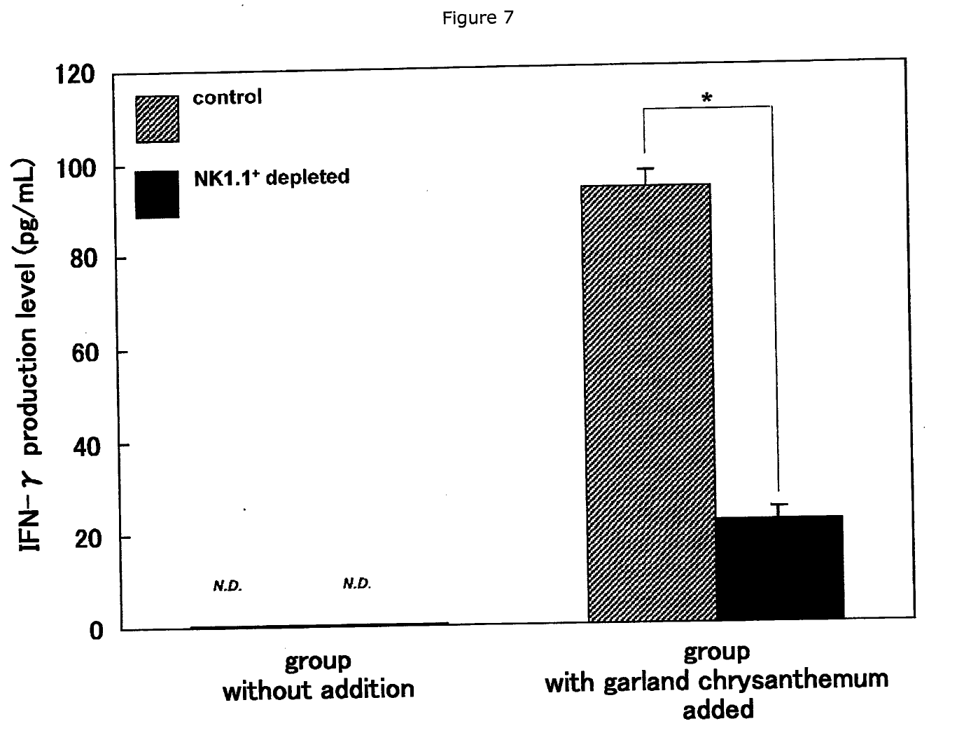

The results are shown in FIG. 7. By comparison to the control, from the fact that the amount of IFN-γ in the spleen cells not containing NK cells and NKT cells was very low, it has been shown that the extract from garland chrysanthemum with 30% by volume ethanol activates NK cells and NKT cells to induce IFN-γ production, and IFN-γ, production of which is induced by the addition of the extract from garland chrysanthemum with 30% by volume ethanol, is mainly derived from NK cells and NKT cells.

### (8) Difference in IFN-γ Induction Activity by Extracting Methods

In order to compare IFN-γ induction abilities in the superheated steam-treated sample of Example (garland chrysanthemum nepurée; “nepurée” is a registered trade mark) and in the ordinarily heat-treated sample in Comparative Example 2 (garland chrysanthemum purée), experiments were carried out using each extract with 30% by volume ethanol in the same way as (1).

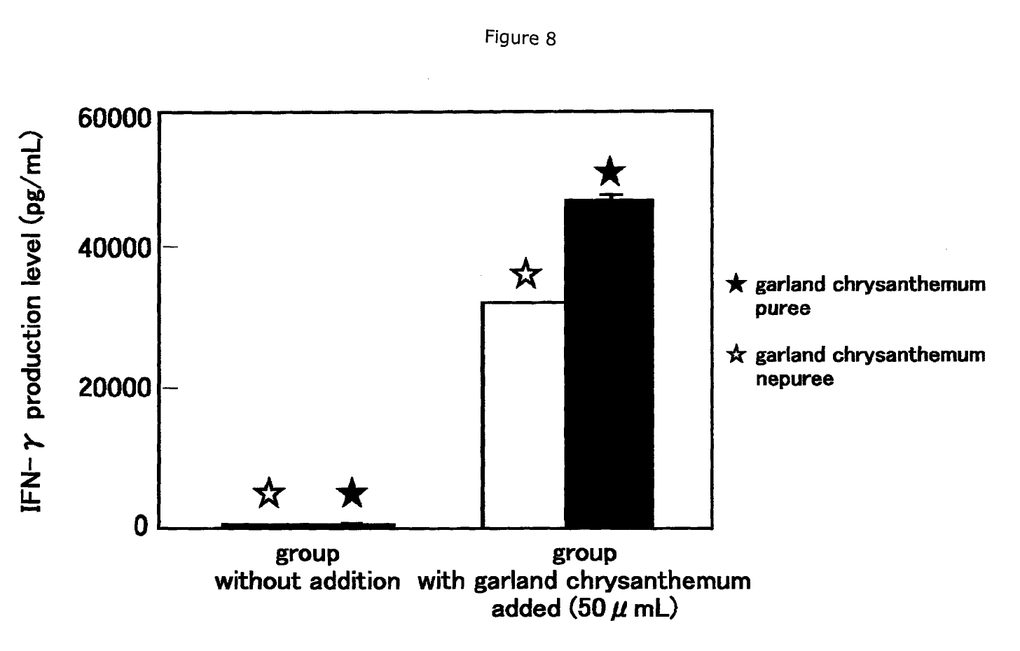

As the result, a stronger activity was demonstrated in nepurée (registered trade mark) as shown in FIG. 8. From this result, it has been shown that a substance which induces the production of IFN-γ is contained naturally in garland chrysanthemum, and the activity is further enhanced by superheated steam treatment.

1. An immune balance regulating agent containing a preparation obtained by superheated steam treatment of garland chrysanthemum.

2. The immune balance regulating agent according to claim 1, which is used for anti-infectious disease.

3. The immune balance regulating agent according to claim 1, which is used for anti-tumor.

4. The immune balance regulating agent according to claim 1, which is used for enhancing type 1 immune system function.

5. The immune balance regulating agent according to claim 4, which is used for dendritic cell activation.

6. The immune balance regulating agent according to claim 4, which is used for promoting IFN-γ and/or interleukin 12 production.
